(19) United States
(12) Patent Application Publication
(10) Pub. No.: US 2022/0348978 A1
(43) Pub. Date: Nov. 3, 2022

(54) Detection of waterborne pathogens with paper strips

(71) Applicant: Sushanta Kumar Mitra, Waterloo (CA)

(72) Inventor: Sushanta Kumar Mitra, Waterloo (CA)

(21) Appl. No.: 17/245,052

(22) Filed: Apr. 30, 2021

(51) Int. Cl. C12Q 1/10; G01N 33/18; G01N 21/78

(52) U.S. Cl. CPC C12Q 1/10; G01N 33/18; G01N 21/78; G01N 2021/7759

(57) ABSTRACT

The disclosure provides methods and apparatuses to test for E. coli in liquid samples. The apparatus includes a paper strip having a hydrophobic area at one end and a reaction area at the other end.

### FIELD

The present disclosure relates generally to the field of detecting pathogens in liquid samples, and, more particularly, to methods and apparatuses for detecting E. coli in water samples using paper substrates.

### BACKGROUND

Routine testing of microbiological water quality is very important, for the safety of public health, due to increased risk of water-borne diseases¹⁻⁶. Some presently known methods for evaluating the quality of water are laboratory-based, tiresome and time-consuming^{1,7-12.} Moreover, they are expensive and require trained technicians to perform the test¹³⁻¹⁵. Therefore, testing of water samples for bacterial contamination is not performed on a daily basis, and the water suppliers and regulators do it in annual or semiannual basis or whenever it required^{1,16}. This kind of minimal evaluation does not ensure the quality of water sources. Hence, field test kits are developed to simplify the testing procedures^{15,17}. These field test kits can be used to test the water samples for assessing microbial quality of water at the point of source itself. In both traditional laboratory-based methods and present field test kits, there is a need for the certain type of instrumentation like an incubator and the time to results varies from 8 hours to 48 hours¹⁸⁻²³ It is desired to reduce the wait times to get the test results for early prevention of water borne diseases.

A litmus test is an indicative test that is used in chemistry to find the general acidity or alkalinity of the substance (liquid or gas) using litmus paper. A litmus paper is made of a dye based on lichens and it turns pink or red in an acid (pH <6.0) whereas it turns blue in a base (pH >8.0). There will be no color change in a solution with pH between 6.0 and 8.0. Litmus paper is inexpensive and is used to differentiate acids and bases. Similar kind of inexpensive litmus tests is not available in biology to identify or detect the biomolecules of interest for samples being tested.

Recent developments in paper-based biosensing technology have opened up an era of creating simple and low-cost rapid detection devices [30-34, and 39]. Most of the paper-based biosensors use the antigen-antibody interactions to detect the target analytes of interest in water, soil, urine, blood or saliva samples [31-34]. Applications built on paper based sensing technology are numerous ranging from testing of blood samples for infectious diseases, testing of grains in agriculture to testing of chemical contaminants in water and soil [30-34 and 39]. Hossain et al. [35] developed a paper-based microfluidic device to detect presence/absence of bacteria using chromogenic substrates. The bacteria in water samples is pre-concentrated using antibody-coated immune-magnetic nanoparticles and then tested the concentrated samples with the paper-based microfluidic device. They detected 5-20 CFU/mL within 30 min using a paper-based system without culturing step and then detected 1 CFU/100mL in 8 hrs with a culturing step. The use of nanoparticles and culture steps bring the complexity of the detection method. Ma et al. [40] developed an immunoassay based paper chips for detecting bacteria in water distribution system. Paper chips used for their work were fabricated by patterning the structures with wax pencil drawing and screen printing method. Further, they implemented the sandwich immunoassay procedure on the patterned areas for detecting E. coli bacteria. The use of antibody immobilization, blocking, immunology reaction and signal amplifications steps bring the complexity of the detection system. Recently, Silver Lake Research Corporation (Azusa, Calif., USA) released a product, Watersafe rapid bacteria test, that detects E. coli in water samples within 15 minutes. The product is based on antigen-antibody interaction on paper strips similar to lateral flow tests. Water quality is evaluated by dipping the paper strip in contaminated water. The formation of two color bands on the paper strip represents the existence of E. coli in water samples. Even though these paper strips are simple to use, inexpensive and rapid but they are not specific to E. coli, fecal, or total coliform, and detects other non-coliform bacteria too.

Accordingly, an improved method and apparatus for detecting E. coli is desired.

### SUMMARY

Recently, Gunda et al.²⁴⁻²⁸ developed two versions of test kits for rapid detection of indicator bacteria, Escherichia coli (E. coli), in water samples within in one hour. However, these test kits require a housing of filter papers and chemicals within plastic enclose to perform the simultaneous concentration and detection of bacteria. These new test kits are simple to use, portable and low cost, however, they may create the problem of disposing of the plastic after testing without affecting the environment.

Recent developments in paper-based biosensing technology have opened up an era of creating simple and low-cost rapid detection devices²⁹⁻³⁴. Most of the paper-based biosensors use the antigen-antibody interactions to detect the target analytes of interest in water, soil, urine, blood or saliva samples³¹⁻³⁴. Applications built on paper based sensing technology are numerous ranging from testing of blood samples for infectious diseases, testing of grains in agriculture to testing of chemical contaminants in water and soil²⁹⁻³⁴. Hossain et al.³⁵ developed a paper-based microfluidic device to detect presence/absence of bacteria using chromogenic substrates. The bacteria in water samples is pre-concentrated using antibody-coated immune-magnetic nanoparticles and then tested the concentrated samples with the paper-based microfluidic device. They detected 5-20 CFU/mL within 30 min using a paper-based system without culturing step and then detected 1 CFU/100 mL in 8 hrs with a culturing step. The use of nanoparticles and culture steps bring the complexity of the detection method. Recently, Silver Lake Research Corporation (Azusa, Calif., USA) released a product, Watersafe rapid bacteria test, that detects E. coli in water samples within 15 minutes. The product is based on antigen-antibody interaction on paper strips similar to lateral flow tests. Water quality is evaluated by dipping the paper strip in contaminated water. The formation of two color bands on the paper strip represents the existence of E. coli in water samples. These paper strips are simple to use, inexpensive and rapid. Conversely, these paper strips are not specific to E. coli, fecal, or total coliform, but detects other non-coliform bacteria too. Moreover, the sensitivity of this kind of paper devices are debatable. Also, the accuracy of these test kits are uncertain and do not have good repeatability because these paper strips use the low sample volume for detection. These paper strips are not following the United States Environmental Protection Agency (US EPA) standards of 100 mL sample volume. Besides, paper-based devices require a different kind of paper strips assembly, reagents, and microparticles to manufacture them.

In the examples set forth herein, the inventors developed a simple and low-cost novel strip that can detect E. coli bacteria within 100 mL volume water samples. In one embodiment, the inventors implemented the E. coli detection capabilities of Mobile Water Kit (MWK)^{27,28} on paper strips. In some embodiments, the paper strip is made of a Grade GB003, Whatman absorbing gel blotting paper with one edge is coated with wax hydrophobic barrier and another edge (attraction zone) is coated with D-glucose (dextrose) solution. There is a reaction zone immediately below the hydrophobic barrier on a paper strip, and it is coated with enzymatic substrates and other ingredients (similar to the chemicals used in MWK and plunger tube assembly) for bacteria detection. The D-glucose coated edge of the paper strip needs to dip into the contaminated water for detecting E. coli bacteria. Sugar acts as a chemoattractant to attract the bacteria in water samples towards the paper strip³⁶ and then water along with attracted bacteria flow though the paper strip towards the reaction zone and then stops at the wax hydrophobic barrier. The use of blotting paper allowed a uniform capillary movement of water along with bacteria towards reaction zone without any requirement of additional pumps.

### BRIEF DESCRIPTION OF THE DRAWINGS

The foregoing and other aspects of the invention will become more apparent from the following description of specific embodiments thereof and the accompanying drawings which illustrate, by way of example only, the principles of the invention. In the drawings:

FIG. 1 shows a schematic of an embodiment of the E. coli detection device;

FIG. 2 illustrates a comparison of an embodiment of the E. coli detection device tested with deionized water (a) and with E. coli contaminated water (b). It is to be noted that the appearance of color on the used E. coli detection device represents the presence of E. coli;

FIG. 3 shows the development of color in samples with decreasing concentrations of E. coli (CFU/100 mL) tested with an embodiment of the E. coli detection device, after 2 hours;

FIG. 4 shows a comparison of response times for an embodiment of the E. coli detection device in the appearance of pinkish red color with various known concentrations of E. coli spiked water samples;

FIG. 5 shows a comparison of the coloration on MWK strips according to an embodiment after 90 minutes when E. coli concentration is at (a) 10⁶ CFU/mL and (b) 10⁵ CFU/mL;

FIG. 6 shows a comparison of MWK strips according to an embodiment;

FIG. 7 shows the uneven nature of the wax coating on paper according to an embodiment;

FIG. 8 shows the color observed on an embodiment after having been dipped in water with an E. coli concentration of approximately 10⁵ CFU/mL;

FIG. 9 shows the color observed on an embodiment after having been dipped in water with an E. coli concentration of approximately 10⁶ CFU/mL; and

FIG. 10 shows the color observed on an embodiment after having been dipped in water with lower and higher concentrations of E. coli after 1 hour.

FIGS. 11A to 11C illustrate another embodiment of the E. coli detection device in which FIG. 11A illustrates a scanning microscope image of porous paper matrix of the paper strip in the detection device; FIG. 11B illustrates the detection device after dipping in deionized water at room temperature; and FIG. 11C illustrates the detection device tested with E. coli contaminated water (2 ×10⁴ CFU/mL) at room temperature, with the appearance of colour on the used device representing the presence of E. coli.

FIG. 12 illustrates development of pinkish red color on another embodiment of the E. coli detection device after 2 hrs based on the concentrations of E. coli (CFU/mL) in which the control shows no color as it is dipped in deionized water.

FIG. 13 is a graph illustrating wait (response) times for the appearance of the pinkish red color with respect to various dip times for various known concentrations of E. coli contaminated water samples.

FIG. 14 illustrates an embodiment of the E. coli detection device to test the water sample for the presence of E. coli bacteria.

FIG. 15 illustrates the results for 40 different water samples tested using an embodiment of the E. coli detection device.

### DETAILED DESCRIPTION OF EMBODIMENTS

The description which follows, and the embodiments described therein, are provided by way of illustration of examples of particular embodiments of the principles of the present invention. These examples are provided for the purposes of explanation, and not limitation, of those principles and of the invention. In some instances, certain structures and techniques have not been described or shown in detail in order not to obscure the invention.

As discussed herein, in one embodiment, the inventors have developed a simple, low-cost paper strip, for detecting the E. coli in water samples by performing enzymatic reactions on the porous paper substrate. The inventors implemented the E. coli detection capabilities of Mobile Water Kit (MWK) [Gunda et al., Analytical Methods, 2014, 6, 6236] on paper strips. Blotting paper strips were used for this purpose. A hydrophobic barrier was created at the top edge of the strip using paraffin wax. This was followed by depositing the MWK chemical solution at the top end of the strip (reaction zone), immediately below the hydrophobic barrier. The wax barrier prevented the spread of the chemicals through capillary action. The strips were completely dried inside the fume hood. The opposite end of paper strip is coated with 0.1M D-glucose (dextrose) solution, which acts as a chemoattractant. Paper strip with glucose coated end is dipped in E. coli contaminated water for detecting E. coli bacteria. E. coli in the water sample is attracted towards the paper strip due to chemotaxis mechanism and the E. coli trapped in paper strip will percolate towards the other end of the paper strip due to capillary action. The E. coli bacteria will concentrate at the top edge of the paper strip and react with the MWK chemicals to produce pinkish red color. The appearance of color on paper strip indicates the presence of E. coli in water samples.

In the broadest embodiment, the E. coli detection device comprises a strip of a long narrow piece of cellulose blotting paper, having a chemoattractant (at one edge), a hydrophobic barrier (at the other edge), and custom formulated chemical reagents (at reaction zone immediately below the hydrophobic barrier). In some embodiments, the hydrophobic barrier comprises wax.

When the E. coli detection device is dipped in water, the E. coli in the water sample is attracted toward the paper strip due to a chemotaxic mechanism, followed by the ascent along the paper strip toward the reaction zone due to a capillary wicking mechanism, and finally the capillary motion is arrested at the top edge of the paper strip by the hydrophobic barrier. The E. coli concentrated at the reaction zone of the paper strip will react with custom formulated chemical reagents to produce a pinkish-red color. Such a color change on the paper strip when dipped into water samples indicates the presence of E. coli contamination in potable water.

The performance of some embodiments of the testing device has been checked with different known concentrations of E. coli contaminated water samples using different dip and wait times. This embodiment of the testing device has also been tested with different interfering bacteria and chemical contaminants. The inventors observe that the different interfering contaminants do not have any impact on the functionalities of the E. coli detection device, and it can become a potential solution for screening water samples for E. coli contamination at the point of source.

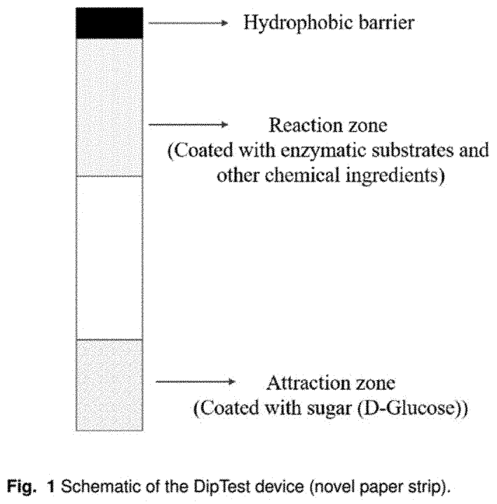

A schematic illustration of an embodiment of the E. coli detection device is provided in FIG. 1.

In this embodiment, the blotting paper (Whatman gel blotting paper, Grade GB003) is diced into 75 mm×5 mm size strips. One edge of the paper strip is coated with wax to form a hydrophobic barrier. The wax barrier prevent the spread of the chemicals and bacteria trapped in reaction zone through capillary action. The reaction zone is formed below the hydrophobic barrier by depositing the 100 ILL of MWK chemical mixture^{27,28}. A person skilled in the art can look into the Gunda et al.^{27,28} paper for more information on MWK chemicals. Later, the other edge of the paper strip is coated with D-glucose (dextrose) by dispensing 100 μL of 0.1 M D-glucose. This edge is also known as attraction zone since D-glucose acts as a chemotaxis agent to attract the E. coli bacteria towards the paper strip in the E. coli detection device. The resulting paper strips were completely dried under a fumehood before dipping them into E. coli contaminated water.

To perform the test, the edge with attraction zone needs to dip into the E. coli contaminated water. The D-glucose in the attraction zone dispersed and forms a concentration gradient in the water. This gradient creates the chemotactic movement of E. coli bacteria from the surrounding water and it eventually increases the migration of bacteria towards the paper strip. The water along with bacteria (attracted at the edge of the paper strip) percolates into the porous matrix of paper strip due to capillary action. Once the water front reaches the hydrophobic barrier on a paper strip. The paper strip will be removed from the water and kept aside on the flat surface. The bacteria trapped in the reaction zone will react with chemicals and produce the pinkish red color. The appearance of pinkish red color indicate the presence of E. coli bacteria.

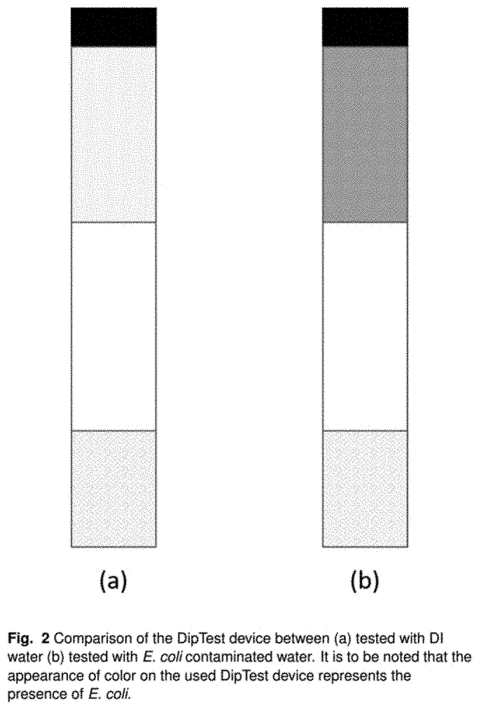

FIG. 2 illustrates the color change on the paper strip of the E. coli detection device because of the presence of a known concentration of E. coli in contaminated water. It is observed that there is a pinkish red color appearing at the reaction zone of the E. coli detection device to represent the presence of E. coli. It is also found that there is no color change on the E. coli detection device with the deionized water to represent there is no bacteria in deionized water sample.

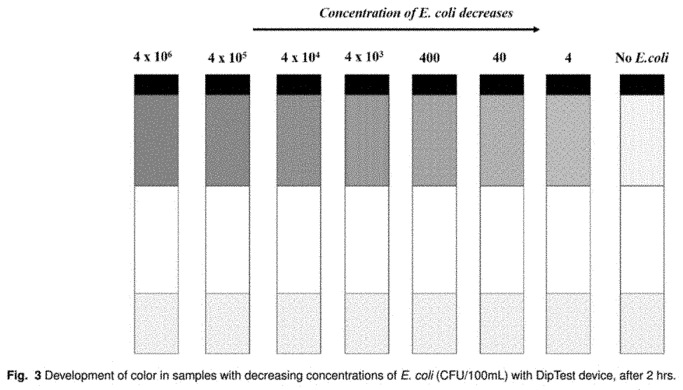

FIG. 3 shows the appearance of pinkish red color at the reaction zone of the E. coli detection device for various known concentrations of E. coli (ATCC 11229) contaminated water samples after 2 hrs of incubation at room temperature. It is to be noted that the color intensity varies based on the concentration of bacteria in water samples and how much time the E. coli detection device is dipped into the water. It is found that the color intensity decreases with the decrease in the concentration of E. coli.

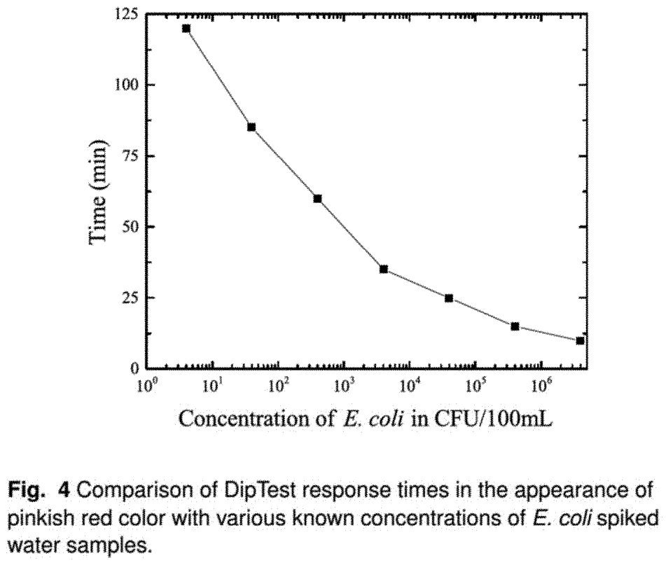

FIG. 4 portrays the comparison of E. coli testing response times in the appearance of pinkish red color at reaction zone with various known concentrations of E. coli spiked water samples. It is observed that the appearance of pinkish red color at reaction zone of E. coli detection device for samples with 4×10⁶ CFU/100 mL to 4×10⁵ CFU/100 mL happens in 10 to 15 min. In one embodiment, the E. coli detection device is expected to detect the 4 CFU/100 mL in 2 hours.

The methods and systems of the present invention are described in the following Examples, which are set forth to aid in the understanding of the invention, and should not be construed to limit in any way the scope of the invention as defined in the claims which follow thereafter.

### EXAMPLE 1—CAPABILITIES OF MWK ON THE MODIFIED DIPTREAT DEVICE

The objective of the experiment was implementing the E. coli detection capabilities of MWK³⁷ on the modified DipTreat³⁸ device to create a simple platform for E. coli/coliform detection in water.

The materials and methods were as follows:

1. Preparation of glucose strips: 200 mL of 0.1M D-glucose (dextrose) solution was prepared under normal laboratory conditions (22° C.). Blotting paper strips (Size: 55 mm×14 mm, Grade GB003, Whatmann®) were completely immersed in the glucose solution and kept on a shaker at 100 rpm for 2 hours. Thereafter, the strips were removed and completely dried (4-5 hours) inside a fume hood. These strips were used in the experiment.

2. Preparation of the MWK chemicals: Refer to the journal article³⁷.

3. Preparation of MWK detection strip: Blotting paper strips (Size: 55 mm×14 mm, Grade GB003, Whatmann®) were used for this purpose. A hydrophobic barrier was created approximately 15 mm from the bottom edge of the strip using paraffin wax coating. This was followed by depositing 200 μL of the MWK chemical solution in the 15 mm by 14 mm space at the bottom of the strip. The wax barrier prevented the spread of the chemicals through capillary action. The strips were completely dried inside the fume hood (3 hours).

4. Device configuration: A glucose strip and a MWK strip was attached end to end on a card board scaffold with double sided tape. This configuration ensured that the portion with the MWK chemicals are in direct contact with the glucose strip.

5. Experimental Procedure: E. coli cells were grown in Lauryl Tryptose Broth (LTB) using previously described techniques³⁸. Different volumes of deionized water was seeded with E. coli to prepare model contaminated water. The bottom portion of the glucose strip of E. coli testing was immersed into sample water and the color change detected over the period of experiment.

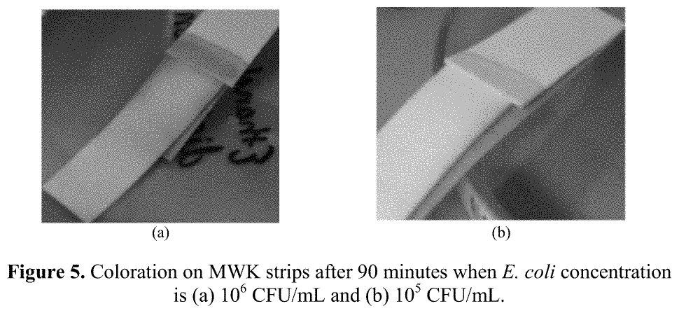

Using 40 mL volumes of sample water with a high bacterial concentration of 10⁵ and 10⁶ CFU/mL, distinct coloration of the MWK strip was observed after 90 minutes of experimental time (FIG. 5). For the higher concentration, the inventors observed color in the bottom glucose strip as well, indicating the leaching of MWK chemicals to the top of the glucose strip in contact with the MWK strip. The first appearance of color for both of these cases was recorded approximately 50 minutes after the start of the experiment. Hence, the E. coli testing can act as a ‘litmus test’ to assess the presence of bacterial contamination. This proof-of-concept provides a new detection method for E. coli on paper strips. Also, it is not restricted to any specific volume of water—as long as there is E. coli in any volume of water, the bottom strip (laced with sugar) will always attract the bacteria, which will travel along the length of the paper strip through capillary action and then react with the MWK chemicals to produce color change.

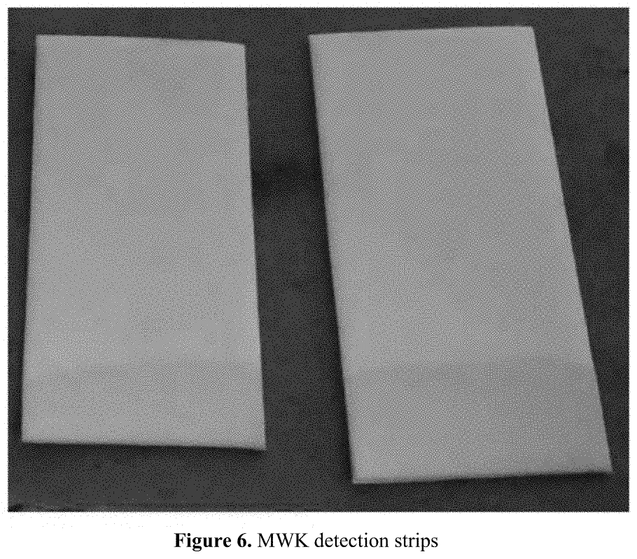

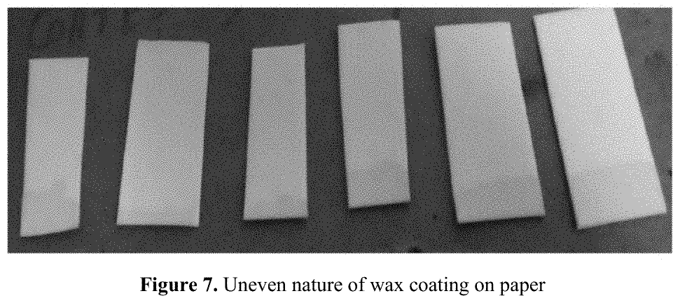

As shown in FIG. 6, MWK detection strips were prepared. Paraffin wax melted on paper strips was used to create a hydrophobic barrier. 200 μL, of MWK chemicals were added to the bottom part. On drying, a yellowish colour was observed. FIG. 7 shows the uneven nature of the wax coating on a batch of prepared paper strips.

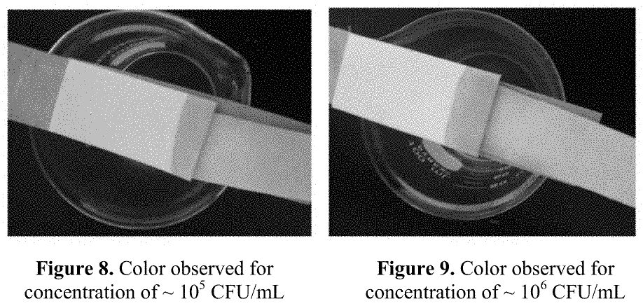

FIGS. 8 and 9 show the colors observed immediately at the end of an experiment where the strips were dipped in water samples with concentrations of E. coli. FIG. 8 shows the color observed for a concentration of approximately 10⁵ CFU/mL, and FIG. 9 shows the color observed for a concentration of approximately 10⁶ CFU/mL.

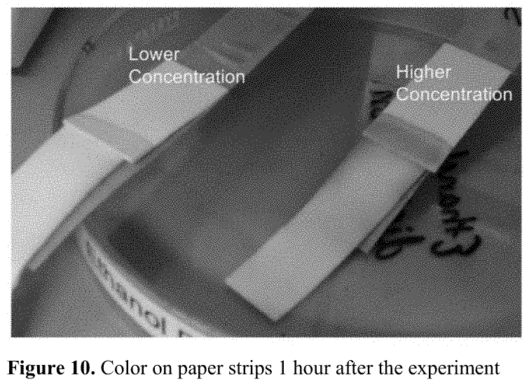

FIG. 10 shows the colors observed after 1 hour. The strip on the left shows the color observed for the lower concentration of E. coli, and the strip on the right shows the color observed for the higher concentration of E. coli.

### EXAMPLE 2—E. COLI TESTING DEVICE

In another embodiment, the inventors used the following materials: Whatman gel blotting paper (0.8 mm thickness, Grade GB003), enzymatic substrate Red-Gal (6-Chloro-3-indolyl-β-D-galactoside) and N,N-Dimethylformamide (DMF) were procured from Sigma Aldrich, Canada. Lauryl Tryptose Broth (LTB) (BD 224150), Bacteria protein extraction reagent (B-PER), Veal Infusion Broth (BD 234420), Bacto 70 Yeast Extract (BD 212750), Brain Heart Infusion Broth (BD 237500), and Nutrient Broth (BD 234000) were purchased from Fisher Scientific, Canada.

Bacteria strains such as E.coli Castellani and Chalmers (American Type Culture Collection (ATCC) 11229), Enterococcus faecalis (E.faecalis) (ATCC 19433), Salmonella enterica subsp. enterica (S. enterica) (ATCC 14028) and Bacillus subtilis (B.substilis) (ATCC 33712, MI112 strain) were obtained from Cedarlane, Burlington, ON, Canada. E.coli K-12 strains were purchased from New England Biolabs, Ipswich, Mass., USA. E.coli ATCC 11229 and E. coli K-12 were grown in LTB medium as well as in nutrient broth medium at 37° C. in incubator (Lab Companion SI-300 Benchtop Incubator and Shaker, GMI, Ramsey, Minn., USA) for 24 hours. B.subtilis bacteria strains were cultured in a growth medium consisting of Veal Infusion Broth and Yeast Extract (5:1 ratio) at 30° C. in incubator for 24 hrs whereas E. faecalis and S. enterica were grown in brain heart infusion broth medium and nutrient broth medium, respectively. Deionized water was used to prepare the respective broth medium.

Broths were sterilized in an autoclave at 121° C. prior to using them for culturing the respective bacteria. Serial dilutions were prepared in deionized water to make bacteria concentrations in the range of 2−2×10⁶ CFU/mL. Water samples with known concentrations of bacteria were utilized to check the performance of E. coli detection device.

Sodium fluoride, EMD ferric chloride (hexahydrate), and EMD sodium chloride were procured from Fisher Scientific, Canada. Sodium nitrate, iron Chloride hexahydrate, ammonia persulfate, sodium iodide, sodium sulfate, potassium hydroxide, sodium bromide, sodium phosphate, and calcium propionate were purchased from Sigma Aldrich, Canada. Standard fluoride solution (1 ppm), fluoride solution (10 ppm), cadmium and lead were obtained from Hanna instruments, Woonsocket, R.I., USA

### EXAMPLE 3—PREPARATION OF CUSTOM FORMULATED CHEMICAL COMPOSITION

In this embodiment, the inventors formulated a new chemical composition by dissolving 100 mg of solid media (1:1 mixture of LTB and Red-Gal) in 4 mL of liquid media (1:2:5 mixture of DMF, B-PER, and deionized water). The enzymatic substrate Red-Gal is used to detect E. coli that secrete β-galactosidase enzymes. A chromogenic compound Red-Gal (6-chloro-3-indolyl-β-D-galactoside) contains two components: 6-chloro-3-indolyl and β-D-galactoside. The β-galactosidase enzyme produced by E. coli hydrolyses this complex Red-Gal molecule resulting in the release of pinkish red color producing dimerized 6-Chloro-3-indolyl compound. The inclusion of B-PER in custom formulated chemical reagents is to accelerate the extraction of β-galactosidase enzymes by lysing the E. coli bacteria cells without denaturing the bacterial enzymes.

### EXAMPLE 4—PREPARATION OF E. COLI DETECTION DEVICE

In this embodiment, the blotting paper is cut into 70 mm×5 mm size strips. While the length of paper strip chosen, i.e., 70 mm, is sufficient for the capillary inhibition to occur, a person skilled in the art will appreciate that other lengths that allow for capillary inhibition to occur can be used in other embodiments. Blotting paper is made of pure cellulose produced entirely from the high quality cotton linters with no additives. In other embodiments, other types of materials known to a skilled person in the art may be used. In this embodiment, the blotting paper has a weight of 320 g/m² , wet strength of 300 mm water column, and water absorbency of 740 g/m². The blotting paper ensures the proper wicking and uniform capillary action.

One edge of the paper strip is coated with wax to form a hydrophobic barrier. The wax barrier prevents the further spreading of the chemicals and bacteria in the reaction zone through capillary action. The reaction zone is formed below the hydrophobic barrier by depositing the 100 μL of above mentioned custom formulated chemical composition (Red-Gal, B-PER and LTB) using pipette and followed by drying under normal laboratory condition (temperature around 23° C.) for one hour. After coating the paper strip with the custom formulated chemical composition in the reaction zone, the opposite edge of the paper strip is coated with D-glucose (dextrose) by dispensing 100 μL of 0.1 M D-glucose and then allowed to be dried at room temperature (23° C.) for one hour. This edge is also known as attraction zone since D-glucose acts as a chemotaxis agent to attract the bacteria towards the paper strip. The resulting paper strips were completely dried for one hour under a fume hood before dipping them into E. coli contaminated water.

### EXAMPLE 5—TESTING WATER SAMPLES WITH E. COLI DETECTION DEVICE

The inventors used an embodiment of the E. coli detection device to test E. coli contaminated water. To perform the test, the edge with attraction zone of E. coli detection device needs to be dipped into the E. coli contaminated water. The D-glucose in the attraction zone gets dispersed and forms a concentration gradient in the water. This gradient creates the chemotactic movement of E. coli bacteria from the surrounding water and it eventually increases the migration of bacteria towards the paper strip of the E. coli detection device [39]. The water along with bacteria (attracted at the edge of the paper strip) percolates into the porous matrix of the paper strip due to capillary action. Once the water front reaches the hydrophobic barrier on the paper strip of the E. coli detection device, the paper strip is removed from the water and kept aside on a flat surface. The bacteria trapped in the reaction zone will react with chemicals and produce the pinkish red color. The appearance of pinkish red color indicates the presence of E. coli bacteria. These tests were conducted at room temperature.

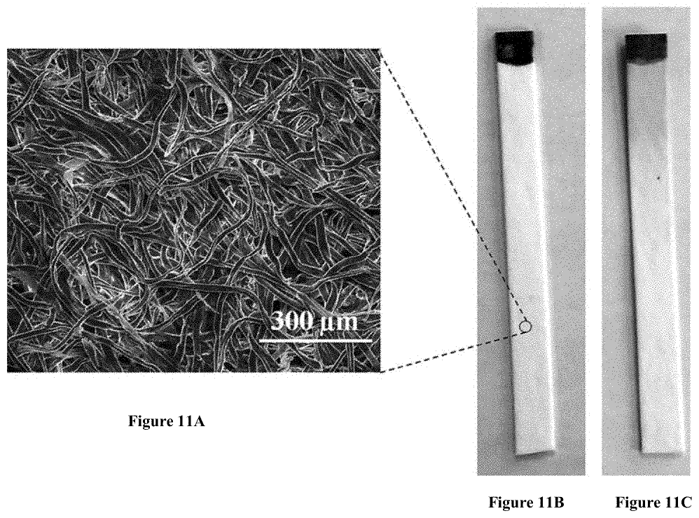

FIGS. 11B and 11C illustrate the color change at the reaction zone of E. coli testing device because of the presence of a known concentration of E. coli (ATCC11229) in contaminated water. The inventors observed that there is a pinkish red color at the reaction zone of the device, which represents the presence of E. coli. The inventors conducted a controlled study where the E. coli detection device was tested in deionized water at room temperature with no E. coli and it is found that there is no color change in the reaction zone.

FIG. 11A shows the scanning electron microscope image of the porous paper matrix of the paper strip used in this embodiment of the E. coli detection device. In this embodiment, the paper is randomly distributed network of paper fibres with an estimated porosity of 65% to 73%.

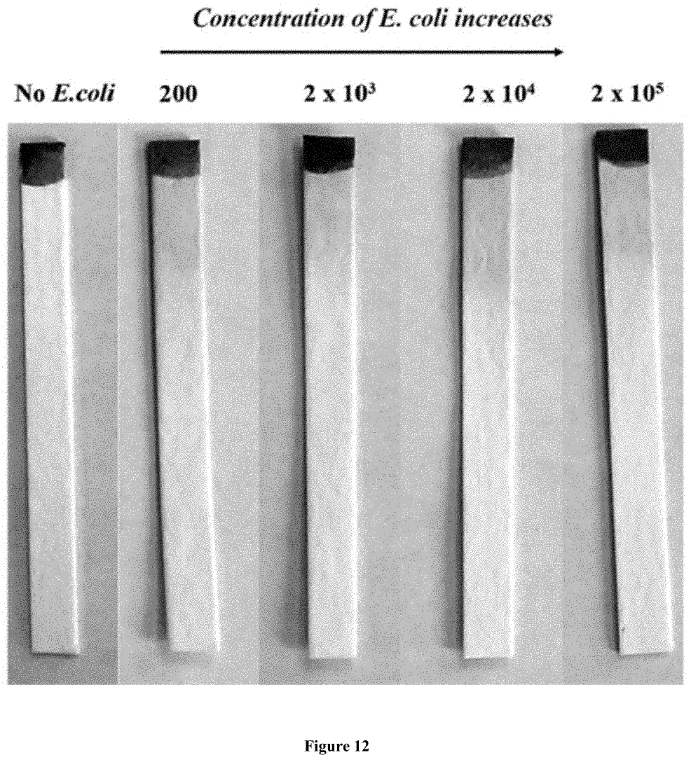

FIG. 12 shows the appearance of pinkish red color at the reaction zone of the E. coli testing device for various known concentrations of E. coli (ATCC 11229) contaminated water samples after 2 hrs at room temperature. It is to be noted that the color intensity varies based on the concentration of bacteria in water samples and how much time the E. coli detection device is dipped into the water. It is found that the color intensity decreases with the decrease in the concentration of E. coli.

The performance of the E. coli detection device is evaluated based on the dip time and wait time. Dip time is the amount of time the E. coli detection device is immersed in the water samples whereas wait time (response time) is the amount of time one has to wait for the results (appearance of pinkish red color) after removing the E. coli detection device from water samples.

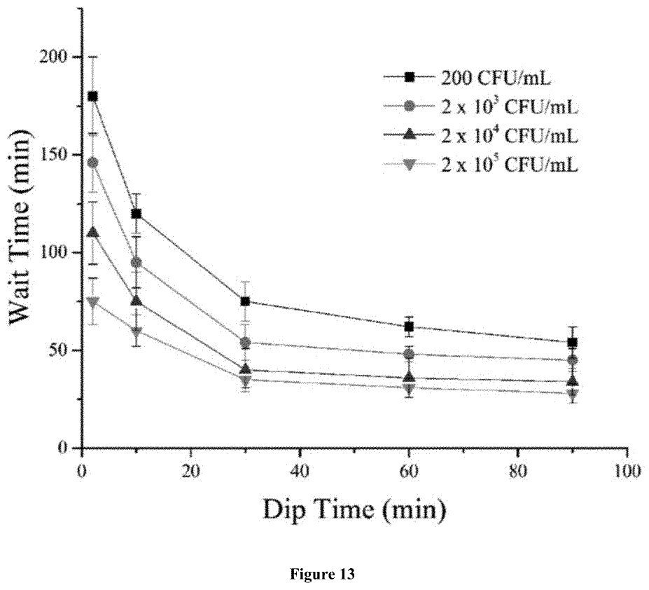

FIG. 13 portrays the comparison of wait (response) times for the appearance of pinkish red color at reaction zone at various dip times and for different known concentrations of E. coli spiked water samples. The average wait times with error bars are provided in FIG. 13. It is observed that the appearance of pinkish red color at reaction zone of E. coli testing device for samples with 2×10⁵ CFU/mL to 4×10⁴ CFU/mL happens in 60 to 65 min (wait time) corresponding to a dip time of 2 min.

The inventors observed wait time decreases with the increase in dip times. The increase in dip time allows a higher number of E. coli bacteria to accumulate at the reaction zone, which in turn decreases the wait time to produce the color due to presence of E. coli bacteria. It is also found that the lower concentrations of E. coli in contaminated water samples take longer wait (response) times compared to higher concentrations of E. coli.

In this embodiment, the space between attraction zone and reaction zone will not influence the performance of the E. coli detection device if the paper strip is kept within the contaminated water samples for a sufficient period of time. In some embodiments, the paper strip is at an optimal length that is required to maintain the stability of the paper strip to sustain the water absorbency for longer time. In some embodiments, the length of the paper strip is 70 mm.

The wicking of E. coli contaminated water into porous paper matrix follows the Washburn-Lucas equation and it is given as [43-48],

L²=γDt/4η* (1)

where, L is the distance moved by the fluid front, γ is the effective surface tension (which includes the effect of any contact angle dependency), D is the average pore diameter of paper, t is the time and η* is the effective viscosity of E. coli contaminated water. Effective viscosity depends on the concentration of E. coli bacteria. The effective viscosity of E. coli contaminated water is provided as [49]:

$${η}^{*} = η [ 1 + 2 ϕ - \frac{c}{2 {πηϵ}_{0}} \frac{1 + 2 λ ( 2 + λ )}{{( 1 + λ )}^{3}} ϕ ] \qquad ( 2 )$$

where, η is the viscosity of water without E. coli bacteria, ϕ is volume fraction occupied by E. coli bacteria in water, ε₀ is the amplitude of the strain rate, c is the point force representing the flagellum, λ is the length of the run between tumbles, representing bacteria motility. By neglecting the motility effects, one can obtain the effective viscosity of the E. coli contaminated water as:

η*=η[1+2ϕ] (3)

E. coli bacteria are usually in rod-shaped and are about 0.25-1.0 μm in diameter and 2.0 μm long, with a bacterial volume of 0.6-0.7 μm³ [50]. Based on the concentrations of bacteria (2×10⁵ CFU/mL to 200 CFU/mL) used in these examples, the volume fraction occupied by E. coli bacteria in water varies from 1.4×10⁻⁷ to 1.4×10⁻¹°, which in turn dictates that there is negligible effect of bacterial suspensions on the viscosity of the contaminated water. Therefore, for further analysis, one needs to decouple the hydrodynamic effects from the reaction kinetics responsible for the appearance of the pinkish red color on the paper strips. The initial rate of interaction of Red-Gal substrate with β-galactosidase enzyme can be described by the Michaelis-Menten equation [51],

$${v}_{0} = \frac{{k}_{cat} {E}_{0}}{1 + \frac{{K}_{m}}{S}} \qquad ( 4 )$$

where, k_{cat} is turnover number and Eₒ is concentration of [3-galactosidase enzyme (released from E. coli bacteria), Kₘ is Michaelis constant and S is the concentration of Red-Gal substrate. It is clear that the wait time for color appearance is solely depended on the interaction of the Red-Gal substrate with β-galactosidase enzyme. The presence of B-PER at the reaction zone helped to accelerate the production of β-galactosidase enzyme from E. coli. While the Red-Gal substrate is used in this embodiment, other substrates known to a person skilled in the art may also be used.

### EXAMPLE 6 —EFFECT OF E. COLI GROWTH MEDIUM ON PERFORMANCE OF THE E. COLI TESTING DEVICE

In order to study the effect of E. coli growth medium on performance of the E. coli detection device, the two E. coli bacteria strains ATCC11229 and K-12 were grown in LTB medium as well as in nutrient broth medium. Water samples contaminated with these E. coli bacteria are tested with an embodiment of the E. coli testing device. The inventors observed that the E. coli detection device produced pinkish red color with both kinds of samples. However, the E. coli bacteria cultured in LTB medium generated a high intensity color compared to the bacteria grown in nutrient broth medium.

### EXAMPLE 7—EFFECT OF INTERFERING BACTERIA AND CHEMICAL CONTAMINANTS

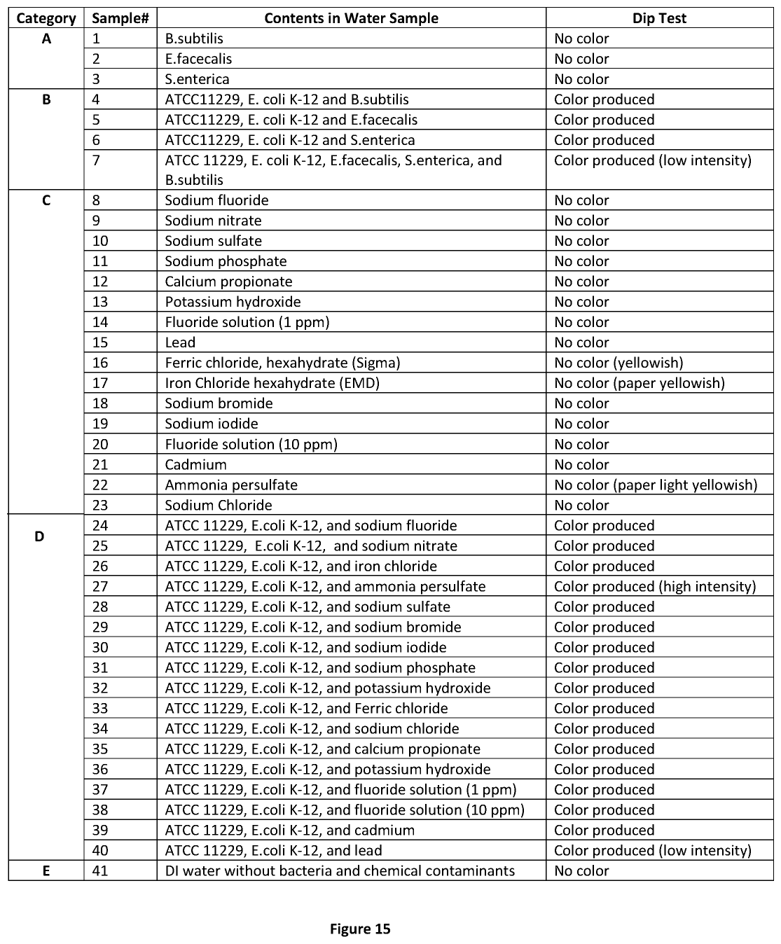

The inventors also tested the effects of interfering bacteria and chemical contaminants on an embodiment of the E. coli detection device. FIG. 15 illustrates the results from using an embodiment of the E. coli detection device to test for 40 different water samples. The E. coli detection device is tested with water samples containing several interfering bacteria. B.subtilis, E. faecalis and S. enterica were used as interfering bacteria.

For Category A (Samples #1-3) water samples, i.e., water samples containing only interfering bacteria (B.subtilis, E.faecalis or S.enterica) and without E. coli bacteria do not produce any color. On the other hand, the E. coli testing device produces color for water samples that contain both interfering bacteria and E. coli (i.e., Category B Samples #4-7). The inventors found that the interference bacteria had no effect on the detection of E.coli with the E. coli detection device.

Similarly, the inventors tested the E. coli testing device with water samples containing several kind of chemical contaminants with and without E. coli bacteria. The E. coli detection device does not give any color when it is tested with water samples (Category C, water samples #8-23) containing different chemical contaminants. However, the E. coli detection device is able to produce the color (pinkish red color) when the device is tested with water samples containing E. coli along with different chemical contaminants (Category D, water samples #24-39). On the basis of these results, the embodiment of the E. coli detection device tested do not react with the chemicals (Red-Gal, B-PER and LTB) coated on the E. coli detection device and the contaminants do not interfere with E. coli bacteria when they are interacting with chemicals (Red-Gal, B-PER and LTB) on the E. coli detection device.

Similarly, the inventors tested the E. coli detection device with negative control, i.e., deionized water without having any bacteria and chemical contaminants (Category E Sample #40). The inventors found that the pinkish red color is not produced the E. coli detection device for this negative control. Accordingly, the E. coli detection device functions properly under different kind of water samples for both positive and negative controls as well as with interfering bacteria and chemical contaminants.

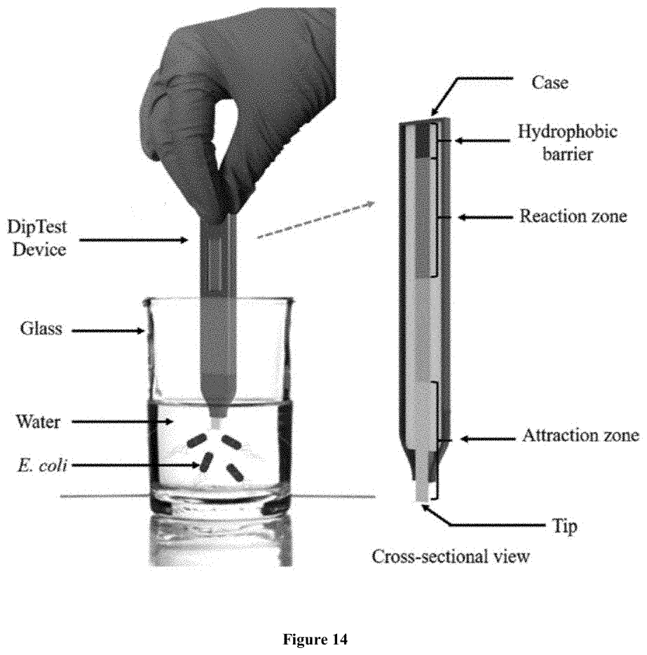

FIG. 14 illustrates the use of an embodiment of the E. coli detection device for the detection of E. coli bacteria in water samples. A user has to dip the E. coli detection device in water for testing purpose. The device can be immersed in water for a certain time and then be removed from the water and placed on a flat surface for the result.

The embodiments of the E. coli detection device as described in this application would be a litmus paper for determining whether the water is safe from bacterial contamination or not. The E. coli detection device is useful in remote locations where one can dip this device and find whether the water is safe to use or not. In particular, the E. coli detection device can be used for checking the quality of water in swimming pools, lakes, rivers, and beaches.

In summary, the inventors have developed a novel E. coli detection device, similar to a litmus test, for detection of E. coli bacteria in water samples. The E. coli detection device can be easily fabricated and simple to use for testing the water samples.

In some embodiments, for a dip time of 2 min, the E. coli detection device is able to detect as low as 200 CFU/mL in 180±20 min and higher concentrations such as 2×10⁵ CFU/mL within 75±12 min. However, for a dip time of 90 min, the E. coli detection device is able to detect as low as 200 CFU/mL in 54±8 min and higher concentrations such as 2×10⁵ CFU/mL within 28±5 min.

Further optimizations in terms of the concentration of individual chemical ingredients used here are needed so that one can eventually have a field deployable device to provide “yes/no” litmus test for E. coli concentration as low as 1-10 CFU/100 mL, thereby meeting the US EPA standards [52].

In some embodiments, the E. coli detection device can be carried in a pocket and test the water samples whenever required. The E. coli detection device can also easily be disposed after completion of test with minimal effort.

In some embodiments, the E. coli detection device can be adapted and integrated with further developments in the detection of other bacteria and pathogens and used not just for water samples but for many other products (milk, wine, juices, etc.) and food industry (frozen meat and cheese).

It is therefore intended that the following appended claims and claims hereafter introduced are interpreted to include all such modifications, permutations, additions, omissions and sub-combinations as may reasonably be inferred. The scope of the claims should not be limited by the preferred embodiments set forth in the examples, but should be given the broadest interpretation consistent with the description as a whole.

Although the invention has been described with reference to certain specific embodiments, various modifications thereof will be apparent to those skilled in the art without departing from the spirit and scope of the invention. Furthermore, it should be understood that the empirical results described previously were provided for the purposes of explanation, and not limitation, of the present invention.

### REFERENCES

1. W. H. Organization, Guidelines for drinking-water quality: recommendations, World Health Organization, 2004, vol. 1.
2. W. Köster, T. Egli, N. Ashbolt, K. Botzenhart, N. Burlion, T. Endo, P. Grimont, E. Guillot, C. Mabilat, L. Newport et al., Assessing microbial safety of drinking water, 2003, 237.
3. R. Barrell, P. Hunter, G. Nichols et al., Commun Dis Public Health, 2000, 3, 8-13.
4. S. T. Odonkor and J. K. Ampofo, Microbiology research, 2013, 4, 2.
5. S. N. Garcia, R. L. Clubbs, J. K. Stanley, B. Scheffe, J. C. Yelderman and B. W. Brooks, Chemosphere, 2013, 92, 38-44.
6. W. Grabow, Water S. A., 1996, 22, 193-202.
7. N. F. Gray, Drinking water quality: problems and solutions, Cambridge University Press, 2008.
8. M. Grant, Applied and Environmental Microbiology, 1997, 63, 3526-3530.
9. R. Lifshitz and R. Joshi, Environmental Toxicology and Water Quality, 1998, 13, 157-164.
10. R. T. Noble, S. B. Weisberg, M. K. Leecaster, C. D. McGee, K. Ritter, K. O. Walker and P. M. Vainik, Environmental Monitoring and Assessment, 2003, 81, 301-312.
11. R. T. Noble, M. K. Leecaster, C. D. McGee, S. B. Weisberg and K. Ritter, Water Research, 2004, 38, 1183-1188.
12. R. Bain, R. Cronk, J. Wright, H. Yang, T. Slaymaker and J. Bartram, PLoS Med, 2014, 11, e1001644.
13. W. H. Organization et al., Assessing Microbial Safety of Drinking Water Improving Approaches and Methods: Improving Approaches and Methods, OECD Publishing, 2003.
14. H. Schraft and L. Watterworth, Journal of microbiological methods, 2005, 60, 335-342.
15. R. Bain, J. Bartram, M. Elliott, R. Matthews, L. McMahan, R. Tung, P. Chuang and S. Gundry, International journal of environmental research and public health, 2012, 9, 1609-1625.
16. J. Bartram, Water safety plan manual: step-by-step risk management for drinking-water suppliers, World Health Organization, 2009.
17. J. Stocker, D. Balluch, M. Gsell, H. Harms, J. Feliciano, S. Daunert, K. A. Malik and J. R. van der Meer, Environmental science & technology, 2003, 37, 4743-4750.
18. A. Rompre, P. Servais, J. Baudart, M.-R. de Roubin and P. Laurent, Journal of microbiological methods, 2002, 49, 31-54.
19. P. Yager, T. Edwards, E. Fu, K. Helton, K. Nelson, M. R. Tam and B. H. Weigl, Nature, 2006, 442, 412-418.
20. R. Chavali, N. S. K. Gunda, S. Naicker and S. K. Mitra, Analytical Methods, 2014, 6, 6223-6227.
21. N. S. K. Gunda, S. Naicker, M. Ghoraishi, subir Bhattacharjee, T. Thundat and S. K. Mitra, 224th ECS Meeting, Oct. 27 -Nov. 1, 2013, San Francisco, Calif., USA, 2013, 2788-2788.
22. N. S. K. Gunda, S. Naicker, M. S. Ghoraishi, S. Bhattacharjee, T. G. Thundat and S. K. Mitra, ASME 2013 11th International Conference on Nanochannels, Microchannels, and Minichannels, 2013, V001T10A001-V001T10A001.
23. S. K. Mitra, N. S. K. Gunda, S. K. Naicker, S. Bhattacharjee, T. Thundat and M. S. Ghoraishi, Microfluidic Detection of Coliform Bacteria, 2014, U.S. Provisional patent application Ser. No. 62/010,821, filed on Jun. 11, 2014.
24. S. K. Mitra, N. S. K. Gunda, S. Naicker and P. Banerji, Coliform detection and Reporting, 2014, U.S. Provisional patent application Ser. No. 62/007,133, filed on Jun. 3, 2014.
25. S. K. Mitra, N. S. K. Gunda and R. Chavali, Method and Apparatus for Detecting Total Coliform And E.Coli in Potable Water, 2015, U.S. Provisional patent application Ser. No. 62/233,734, filed on Sep. 28, 2015.
26. S. K. Mitra, N. S. K. Gunda and R. Chavali, Method and Apparatus for Detecting Total Coliform And E.Coli in Potable Water, 2015, Indian patent application Serial No. 3096/MUM/2015, filed on Aug. 14, 2015.
27. N. S. K. Gunda, S. Naicker, S. Shinde, S. Kimbahune, S. Shrivastava and S. Mitra, Analytical Methods, 2014, 6, 6236-6246.
28. N. S. K. Gunda, R. Chavali and S. K. Mitra, Analyst, 2016, 141, 2920-2929.
29. C. Parolo and A. Merkoci, Chemical Society Reviews, 2013, 42, 450-457.
30. A. W. Martinez, S. T. Phillips, G. M. Whitesides and E. Carrilho, Analytical chemistry, 2009, 82, 3-10.
31. C.-z. Li, K. Vandenberg, S. Prabhulkar, X. Zhu, L. Schneper, K. Methee, C. J. Rosser and E. Almeide, Biosensors and Bioelectronics, 2011, 26, 4342-4348.
32. R. Fobel, A. E. Kirby, A. H. Ng, R. R. Farnood and A. R. Wheeler, Advanced materials, 2014, 26, 2838-2843.
33. X. Li, D. R. Ballerini and W. Shen, Biomicrofluidics, 2012, 6, 011301.
34. G. A. Posthuma-Trumpie, J. Korf and A. van Amerongen, Analytical and bioana-lytical chemistry, 2009, 393, 569-582.
35. S. Z. Hossain, C. Ozimok, C. Sicard, S. D. Aguirre, M. M. Ali, Y. Li and J. D. Brennan, Analytical and Bioanalytical Chemistry, 2012, 403, 1567-1576.
36. S. Dasgupta, N. S. K. Gunda and S. K. Mitra, Environmental Science: Water Research & Technology, 2016, 2, 931-941.
37. Gunda, N. S. K., Naicker, S., Shinde, S., Kimbahune, S., Shrivastava, S., & Mitra, S. (2014). Mobile water kit (MWK): a smartphone compatible low-cost water monitoring system for rapid detection of total coliform and E. coli. Analytical Methods, 6(16), 6236-6246.
38. Dasgupta, S., Gunda, N. S. K., & Mitra, S. K. (2016). Evaluation of the antimicrobial activity of Moringa oleifera seed extract as a sustainable solution for potable water. RSC Advances, 6(31), 25918-25926.
39. Parolo C, Merkoçi A. Paper-based nanobiosensors for diagnostics. Chemical Society Reviews. 2013; 42(2):450-457.
40. Ma S, Tang Y, Liu J, Wu J. Visible paper chip immunoassay for rapid determination of bacteria in water distribution system. Talanta. 2014; 120:135-140.
41. Mitra S K, Gunda N S K, Dasgupta S. Method and Apparatus for Detecting E. coli with Paper Strips; 2017.
42. Mitra S K, Gunda N S K, Dasgupta S. Method and Apparatus for Detecting E. coli with Paper Strips; 2017.
43. Fu E, Ramsey S A, Kauffman P, Lutz B, Yager P. Transport in two-dimensional paper networks. Microfluidics and Nanofluidics. 2011; 10(1):29-35.
44. Masoodi R, Pillai K M. Wicking in Porous Materials: Traditional and Modern Modeling Approaches. CRC Press; 2012.
45. Cai J, Yu B. A discussion of the effect of tortuosity on the capillary imbibition in porous media. Transport in Porous Media. 2011; 89(2):251-263.
46. Hsu H H. Particle Movement in Paper Porous Media: Influence Factors and Model. McMaster University; 2010.
47. Das S, Mitra S K. Different regimes in vertical capillary filling. Physical Review E. 2013; 87(6):063005.
48. Das S, Waghmare P R, Mitra S K. Early regimes of capillary filling. Physical Review E. 2012; 86(6):067301.
49. Haines B M, Aranson I S, Berlyand L, Karpeev DA. Effective viscosity of dilute bacterial suspensions: a two-dimensional model. Physical Biology. 2008; 5(4):046003.
50. Kubitschek H. Cell volume increase in Escherichia coli after shifts to richer media. Journal of Bacteriology. 1990; 172(1):94-101.
51. Xie D, Suvorov L, Erickson J W, Gulnik S. Real-time measurements of dark substrate catalysis. Protein Science. 1999; 8(11):2460-2464.
52. Olstadt J, Schauer J, Standridge J, Kluender S. A comparison of ten USEPA approved total coliform E.coli tests. Journal of Water and Health. 2007; 5(2):267-282.

All publications mentioned herein are hereby incorporated by reference in their entireties. While the foregoing invention has been described in some detail for purposes of clarity and understanding, it will be appreciated by one skilled in the art, from a reading of the disclosure that various changes in form and detail can be made without departing from the true scope of the invention in the appended claims.

1. Methods comprising any features, combinations of features and/or sub-combinations of features described herein.

2. Apparatuses comprising any features, combinations of features and/or sub-combinations of features described herein.

3. Systems comprising any features, combinations of features and/or sub-combinations of features described herein.
